(19) United States
(12) Patent Application Publication
(10) Pub. No.: US 2014/0155871 A1
(43) Pub. Date: Jun. 5, 2014

(54) METHOD FOR PREPARING CORNEAL DONOR TISSUE FOR REFRACTIVE EYE SURGERY UTILIZING THE FEMTOSECOND LASER

(71) Applicant: James Stuart Cumming, Laguna Beach, CA (US)

(72) Inventor: James Stuart Cumming, Laguna Beach, CA (US)

(73) Assignee: James Stuart Cumming, Laguna Beach, CA (US)

(21) Appl. No.: 13/891,088

(22) Filed: May 9, 2013

Related U.S. Application Data
(60) Provisional application No. 61/688,265, filed on May 10, 2012.

(51) Int. Cl. A61F 9/008; A61F 2/16

(52) U.S. Cl. CPC A61F 9/00834; A61F 2/16
USPC 606/5

(57) ABSTRACT

A method of accurately fabricating a non-human donor corneal tissue for implantation into a recipient human cornea, the method comprising the steps of: removing corneal tissue from a donor with a femtosecond laser; placing the corneal tissue in a fixative solution for a selected time interval to cross-link the collagen fibrils in the tissue and prevent swelling of the corneal tissue; and shaping the tissue to provide a conical inlay of a selected shape and thickness having one or more radial extensions. The method is such that the corneal inlay may be attached to the peripheral corneal and/or the sclera. The method is such that the corneal inlay may be stored for a period of up to two years prior to attachment.

### RELATED APPLICATIONS

This application claims priority to U.S. Provisional Patent Application No. 61/288,265, filed on May 10, 2012, the entire contents and disclosure of which is hereby incorporated by reference.

### BACKGROUND OF THE INVENTION

The correction of refractive errors of the eye by the attachment of donor material directly onto, or into, the cornea or by corneal transplant has been carried out in the prior art for several years, One technique is known as “epikeratophakia”, This technique involves the formation of a ring-shaped trough in the recipient corneal bed, and the placement and suturing of the donor material onto the optical center of the cornea with its edges fitting into the trough. This technique requires that the epithelium of the cornea grow over the resulting lenticule, which attaches the lenticule to the surface of the cornea.

Another prior art procedure for refractive correction of the eye is a technique referred to as “intralamellar insertion”. This latter procedure involves the insertion of a lenticule into a pocket prepared in the recipient's corneal stroma.

Both of the foregoing techniques require a material which transmits light, and which will allow fir the passage of oxygen, glucose, and amino acids for the metabolic and nutritional requirements of the cornea. A lenticule freeze-lathed from human tissue, or from a material of high water content such as hydroxyethylmethylmetha-crylate, has been used for these prior art procedures. However, such materials suffer from unpredictability regarding the final shape of the lenticule, and therefore the refractive correction of the surgical procedure.

Since the corneal stroma is maintained in a partially dehydrated state, by the fluid barrier provided by the corneal epithelium, and the fluid pumping action provided by the corneal endothelium, the lenticule material is also subject to dehydration, which is not totally predictable.

In addition, the initial cutting of the presently used corneal donor material is subject to variation depending upon the water content of the material at the time of freezing and lathing. There is, therefore, a need for a lenticule material and a method of shaping it which can be cut predictably, and which will provide a known lenticule shape regardless of minor variations in the hydration of the donor corneal tissue. There is also a need to be able to measure the optical power of the lenticule before it is sutured into, or placed into a pocket, in the cornea.

Accordingly, a major problem, which is encountered when human corneal tissue is used to form the lenticule its unpredictability, as discussed above. Specifically, it is not presently feasible to determine the refractive power of the resulting lenticule prior to its attachment to the eye of the recipient. This problem is compounded by the fact that if a number of human corneal tissues are lathed to a particular thickness, they tend to exhibit different refractive powers. These problems lead to substantial trial and error in present-day refractive surgical procedures, often requiring multiple attempts to form a lenticule, with the correct refractive power.

Several surgical options for providing near vision in patients who have had cataract surgery have been evaluated with varying degrees of success.

One such surgical solution is monovision. With monovision, one eye is corrected for near vision (using a contact lens, intraocular lens, or LASIK), and the other eye is focused at distance. However, many patients complain of blurred vision, glare and halos resulting in difficulty in driving at night, and loss of depth perception; they are unable to tolerate these side effects.

Another such surgical solution is the implantation of multifocal intraocular lenses (“IOLs”). While a number of multifocal intraocular lenses are commercially available and have been shown to allow good functional vision without the use of corrective lenses, they are also known to cause glare disability, and halos. In at least one clinical trial comparing monovision and multifocal IOLs for presbyopia correction, 4.7% of patients had the multifocal lenses removed, mainly due to dysphotopsia. One doctor noted that in his practice, the debilitating glare side-effect led to 4.5% to 8.5% of multifocal IOL explantation.

Another such surgical solution is the use of corneal inlays as a possible treatment for presbyopia by producing monovision. Although the long-term results of one of these devices demonstrate safety and effectiveness for the correction of presbyopia, 15.6% of patients reported severe night-vision problems at 3 years in one study while in another study 10% were explanted during the study. Additionally, since conical inlays utilize the monovision treatment with a Laser-Assisted in situ Keratomileusis (“LASIK”) procedure, they have the associate risks described above for monovision. Moreover, a major concern with corneal inlays is the potential for deformation of the cornea (keratectasia), which can lead to a severe decrease in vision, which is very difficult to treat.

LASIK is a commonly performed eye surgical procedure used to correct distance vision that requires a flap to be cut almost completely across the cornea. Because the cornea is without a blood supply (except for the 5-7 cell thick superficial epithelial layer) the superficial and posterior components never heal together.

A more recent development in ophthalmic surgery is the Femtosecond laser, which can perform very accurate and, if necessary, precise irregular incisions in the eye tissue. It can also cut accurate sections of tissue, called lenticules, to place into a conical pocket. These lenticules can be cut to a shape that works to strengthen the human cornea or for refractive purposes to treat myopia, presbyopia, correct astigmatism, or for corneal transplantation.

There are two tough membranes stretching across the cornea's structure and sandwiching the stroma: the Bowman's membrane (anterior) and Descemet's membrane (posterior). These two membranes together account for most of the corneal stability and, therefore, maintain the refractive power of the cornea. The cornea is subjected to the internal pressure of the eye, which, should the structural integrity of the cornea be reduced (e.g. by LASIK), can cause a bulging forward of the posterior conical segment with a resulting deformation of the anterior corneal surface, leading to conical ectasia.

It is now becoming apparent that a significant number of patients having had LASIK are developing conical ectasia, sometimes many years after surgery. Corneal ectasia consists of a thinning of the posterior thin remaining cornea, posterior to the anterior flap. The section of the cornea in the LASIK procedure separates the sandwich of Bowman's (the front strong supporting membrane), from Descemet's (the strong posteriorly supporting membrane). The intraocular pressure causes the thin posterior cornea to bulge forward, distorting the anterior flap resulting in irregular astigmatism frequently leading to the need for a conical transplant.

A method of cross linking the human corneal stroma has been developed and has some success in slowing the progression of the ectasia. Cross linking stiffens the cornea by strengthening the bonds between the collagen molecules. Various methods of cross linking are known to those of ordinary skill in the art, and may include saturating an abraised cornea with Ribofalvin (Vitamin B12) and then applying ultraviolet light for a predetermined period of time,

The present invention provides a method of treating corneal ectasia and any condition where there is damage to the human cornea that compromises site. The donor tissue is designed to be thick centrally to support the bulging posterior cornea of patients with ectasia and sutured or glued into peripheral slots in the peripheral cornea and adjacent sclera. This technique can be used to treat ametropia, the complications of LASIK, corneal inlays and keratoconus.

The accurate incisional properties of a Femtosecond laser may be utilized to cut conical pockets, lamellar sections and full thickness irregularly shaped corneal buttons from both a recipient and donor and have them match perfectly for either a penetrating keratectomy, lamellar anterior or posterior grafts and corneal inlays, including the equivalent of the Descemet's membrane in a donor graft that has been treated to prevent rejection by the recipient.

A Femtosecond laser may be used to fashion a strong inlay to traverse the space between the anterior flap and posterior cornea. The laser may be used to cut slots in the peripheral uncut cornea, and the slots can be extended into the adjacent sclera with a knife to fashion slots into which the donor inlay can be sutured or glued.

Donor tissue of various configurations may be cut with the Femtosecond laser obviating the necessity of freezing the donor tissue.

In addition, various cuts in the donor tissue can be designed for full, or partial thickness conical transplants, or for refractive corneal inlays, since an approximate power of the donor inlay can be refined following the initial surgery, at a later date, by the laser.

The corneal inlay may be intentionally made thicker than necessary to stabilize the cornea, so that at a later date it may be refined using the Femtosecond laser, to provide good vision.

The method of the present invention, in one of its embodiments, utilizes specially treated animal corneal tissue, obtained preferably from pigs or sharks. This is suggested, for example, in U.S. Pat, No. 4,346,482, which issued Aug. 31, 1982 in the names of Jerald L. Tennant et al. The specially treated pig, or shark, corneal tissue has a much higher predictable refractive power than the human tissue when cut with the Femtosecond laser to a particular shape and thickness. Moreover, the specially treated pig, or shark, corneal tissue is clearer than human corneal tissue and may be subjected to pre-testing before eye surgery.

Accordingly, the present invention makes possible the creation of a storage bank of donor tissues having pre-established refractive powers, and which may be conveniently selected and used for refractive eye surgery. An important aspect of the invention involves the treatment of the donor tissue with a fixative agent, such as gluteraldehyde, which not only neutralizes the immune response, and cross-links the tissue to prevent swelling, but which also enables the tissues to be stored for long periods of time without deterioration. Another aspect of the invention is the preparation of a cortical inlay with pre-placed sutures to facilitate surgery and reduce distortion and astigmatism.

An objective of the invention is to provide a method utilizing the Femtosecond laser for fabricating the partial or total corneal donor tissue, for use in both corneal repair or refractive eye surgery which is formed from readily available donor tissue, and which comprises the step of treating the Femtosecond cut tissue with a fixative agent such as gluteraldehyde, so that the tissue will not induce a rejection response of the recipient immune system. This additionally enables the tissue to be stored for tong periods of time without degradation.

Another object of the invention is to provide such a method in which the tissue can be prepared in sufficient numbers to meet implantation requirements without the need to await the availability of human donor tissues.

Another objective of the invention is to provide a method for fabricating the corneal inlay to a selected refractive power, which may be established with a high degree of precision prior to the attachment of the corneal inlay to the eye of the patient.

Another objective of the invention is to cut a corneal inlay for correction of astigmatism that has an extension along the axis of the tonic surface of the corneal inlay that can be inserted into a pocket with the peripheral cornea and/or the sclera.

Yet another objective of the invention is to provide a method for fabricating the corneal inlay which does not change shape upon change of the water content during the fabrication process or upon change of the water content of the recipient cornea, and which will maintain its clarity in the eye of the patient.

It is therefore desirable to provide a method for preparing corneal donor tissue for refractive eye surgery that overcomes the disadvantages taught by the prior art.

Other features and advantages of the present invention will become apparent from the following more detailed description, taken in conjunction with the accompanying drawings, which illustrate, by way of example, the principles of the presently described apparatus and method of its use.

### BRIEF DESCRIPTION OF THE SEVERAL VIEWS OF THE DRAWING(S)

Illustrated in the accompanying drawing(s) is at least one of the best mode embodiments of the present invention. In such drawing(s):

FIG. 1 is a flow-chart illustrating a method for preparing corneal donor tissue for refractive eye surgery utilizing a femtosecond laser according to a preferred embodiment of the present invention;

FIG. 2 is a schematic top view of a conical inlay inserted into conical pocket according to a preferred embodiment of the present invention; and

FIG. 3 is a schematic top view of a conical inlay inserted into conical pocket according to a preferred embodiment of the present invention.

### DETAILED DESCRIPTION OF THE PREFERRED EMBODIMENTS

The above described drawing figures illustrate the described invention in at least one of its preferred, best mode embodiment, which is further defined in detail in the following description. Those having ordinary skill in the art may be able to make alterations and modifications to what is described herein without departing from its spirit and scope. Therefore, it should be understood that what is illustrated is set forth only for the purposes of example and should not be taken as a limitation on the scope of the present invention.

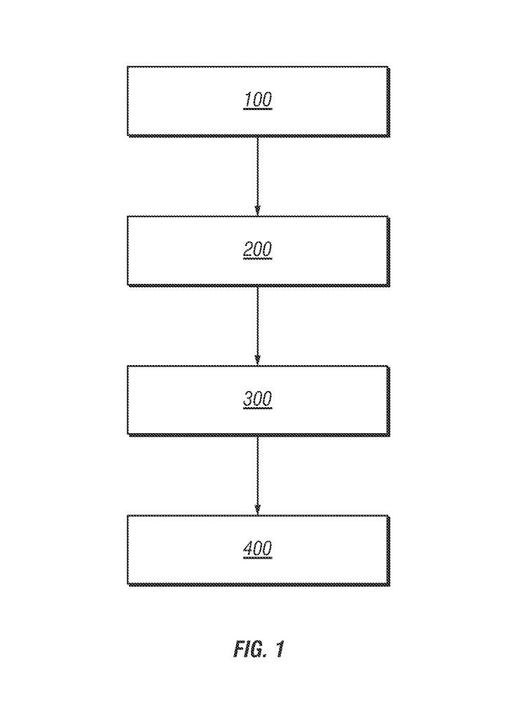

FIG. 1 illustrates a method for preparing cortical donor tissue for refractive eye surgery utilizing a femtosecond laser according to a preferred embodiment of the present invention.

Step 100: Donor corneal tissue is removed from a donor cornea. The donor cornea is preferably selected from a group of animals consisting of: pigs, cows, rabbits, cats, dogs, primates, cetacean dolphins, sharks, and warm blooded animals and chondrychthes. The accurate incisional properties of a femtosecond laser may be used to remove the donor conical tissue from the donor cornea. As discussed herein, due to the accurate incisional properties of the femtosecond laser, the donor corneal tissue may he cut in a shape so as to accomplish the objectives of the present invention discussed herein. Preferably, the donor tissue is designed to he thick near the center.

Step 120: The donor tissue is subjected to fixation in a solution. Preferably, the donor corneal tissue is exposed to the fixation agent very shortly after it is removed from the donor cornea. Fixation in the solution cross-links the collagen fibrils and prevents swelling of the tissue. The solution preferably contains gluteraldehyde. A preferred solution contains 0.5%-4% gluteraldehyde in a 0.1M phosphate buffer. Alternatively, formaldehyde, or other aldehyde groups may be used. Moreover, the fixative may contain acrolein, or other substances, which react with protein or glycoprotein in the donor tissue to prevent an immune response. The time required for fixation varies from tissue to tissue, but a fixation time of up to two weeks is preferred.

Step 200: The donor corneal tissue is washed in a physiological solution, preferably a 0.1M phosphate buffer, so as to remove detectable traces of gluteraldehyde. Preferably, the solution is replaced several times over a period of days. Moreover, in order to prevent bacterial growth, washing the donor corneal tissue in the physiological solution may occur at a controlled temperature, such as four degrees Celsius, in order to inhibit bacterial growth.

Step 300: The donor conical tissue is lathed so as to form a conical inlay. Preferable techniques include freeze-lathing, vacuum-lathing, and lathing using an ablative laser. The ablative laser is preferably a femtosecond or excimer laser at 193 nm, and may be used to shape the donor corneal tissue. This technique results in a corneal inlay surface that is smooth and can rapidly adapt to a recipient cornea upon insertion. The corneal inlay may be fabricated to have desired attributes, such as thicknesses or toricity depending upon the desired attributes of the corneal inlay, such as refractive powers. Moreover, the corneal inlay may be intentionally made thicker than necessary to stabilize the cornea, so that at a later date it may be refined using the Femtosecond laser, to provide good vision. Indeed, the corneal inlay may be formed with the appropriate attributes to correct myopia, hyperopia, presbyopia, astigmatism, or any combination thereof.

The corneal inlays may be stored in a solution of four-percent (4%) formaldehyde for long periods of time without degradation so as to enable preparation in sufficient numbers to meet implantation requirements without the need to await the availability of human donor tissues. Such long periods of time include periods of up to two years in a sterile environment. However, longer periods may be attainable.

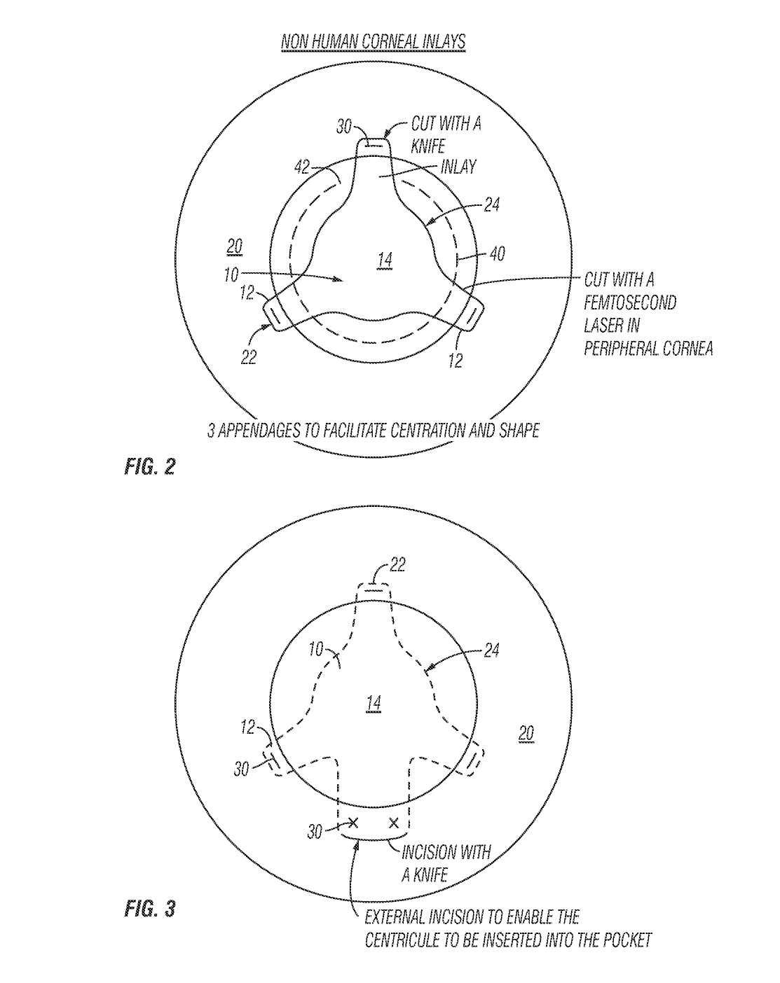

As illustrated in FIGS. 2 and 3, the corneal inlay 10 (or graft or corneal inlay) may be lathed (or cut) so as to have one or more extensions 12 along the axis of the tonic surface 14 of the conical inlay. The corneal inlay may be inserted into a corneal pocket 24—preferably cut with the femtosecond laser to accept the corneal inlay. The extension may be inserted into a scleral pocket 22 in the peripheral cornea and/or the sclera 20. The scleral pocket may be cut with a knife.

The extensions may be inserted into the peripheral cornea and the surrounding sclera to support an ectasic posterior corneal layer caused, for instance, by section of the cornea following LASIK or corneal inlay procedure. The corneal inlay may be fabricated to treat both regular and irregular astigmatism, and to correct the corneal astigmatism, or any of the conditions described herein. The extensions may be fabricated to be sutured or glued into the peripheral cornea and adjacent sclera to maintain the donor tissue on-axis with the recipient's cornea. These extensions facilitate centration and maintain the shape of the corneal inlay. This is particularly useful because the cortical inlays of the present invention do not change shape upon a change in the water content, e.g. during the fabrication process, within the recipient cornea, and/or during storage. Thus, the corneal inlay will maintain its clarity in the eye of the recipient. Moreover, the shape of the corneal inlay, once inserted, prevents the deformation of the anterior corneal flap, i.e. reduces the aforementioned bulge, which in turn affects optical power.

As illustrated in FIG. 2, the conical inlay (or graft) may comprise regularly spaced radial extensions. For example, three such extensions may be cut from the donor cornea tissue during the corneal inlay formation process described herein. Preferably, for the reasons discussed herein, the femtosecond laser is used to form the corneal inlay and its extensions. The corneal inlay is formed so as to be inserted into the corneal pocket, the extensions sutured to the sclera. In at least one embodiment, extensions are sutured or glued to a scleral pocket 22 cut into the sclera 20. Accordingly, the donor tissue may remain on-axis with the recipient's cornea. As used herein, the term on-axis refers to the alignment of the optical axis of the cornea and that of the corneal inlay. In FIGS. 2 and 3, that axis would be normal to the page. Alternatively, as illustrated in FIG. 3, the conical inlay may comprise irregularly spaced radial extensions. However, due to a perceived minimal amount of distortion effects, a three-extension corneal inlay is preferred, The shaping of the corneal inlay is made possible through the use of the femtosecond laser. Indeed, a variety of corneal inlay shapes may be had so as to accomplish a variety of desired features.

As discussed herein, the conical inlay is formed so as to be inserted into the corneal pocket with the extensions being sutured to the scleral pocket. The sacral pocket may be an incision formed within the sclera to accept a distal end of one of the extensions. Prior to insertion of the extension into the scleral pocket, the scleral pocket may be held open with viscoelastic material, such as Healon® or the like.

Step 490: Sutures 30 are pre-placed in the corneal inlay (or graft) so as to enable more ready application to the recipient's cornea. Pre-placing sutures may be accomplished far more readily during manufacture than in the surgical suite, and results in a symmetrical pattern of ties. This permits the corneal inlay to be adjusted when fastened to the recipient surface without introducing undesired astigmatism.

The Femtosecond laser may be used to shape one or both surfaces of the corneal inlay and the recipient's cornea. The corneal inlay may be fabricated with a cylindrical, as well as spherical correction, if so desired, which may correct astigmatism.

In addition to the conditions discussed herein, the present invention may be utilized for treating keratoconus and kerato ectasia.

The enablements described in detail above are considered novel over the prior art of record and are considered critical to the operation of at least one aspect of the invention and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that the named inventor believes that the claimed subject matter is what is intended to be patented.

What is claimed is:

1. A method of accurately fabricating a non-human donor conical tissue for implantation into a recipient human cornea, the method comprising the steps of:
removing cortical tissue from a donor with a femtosecond laser;
placing the corneal tissue in a fixative solution for a selected time interval to cross-link the collagen fibrils in the tissue and prevent swelling of the corneal tissue; and
shaping the tissue to provide a conical inlay of a selected shape and thickness having one or more radial extensions.

2. The method of claim 1, wherein the fixative solution comprises gluteraldehyde.

3. The method of claim 1, wherein the fixative solution comprises 0.1 M phosphate buffer.

4. The method of claim 1, wherein the fixative solution comprises 0.5% to 4.0% gluteraldehyde in 0.1 M phosphate buffer.

5. The method of claim 1, wherein the selected time interval in which the tissue is placed in the fixative solution is up to two weeks.

6. The method of claim 1, further comprising the intermediate step of:
washing the corneal tissue in a physiological solution prior to shaping,

7. The method of claim 1, further comprising the intermediate step of:
attaching sutures to the conical inlay after shaping so as to reduce astigmatism when the conical inlay is attached to a human sclera.

8. The method of claim 7, wherein the sutures are attached to the radial extensions.

9. The method of claim 1, wherein the corneal tissue is shaped while in a non-frozen state.

10. The method of claim 1, wherein the corneal tissue is shaped using the femtosecond laser.

11. The method of claim 1, wherein the corneal tissue is that of an animal selected from the group of: pigs, cows, rabbits, cats, dogs, primates, cetacean dolphins, sharks, mammals and chondrychthes,

12. The method of claim 1, wherein the corneal tissue may be in the form of a partial or full thickness graft.

13. The method of claim 1, wherein the extensions may be inserted into the sclera of the recipient cornea so as to support an ectastic posterior cortical layer of the recipient cornea.

14. The method of claim 13, wherein the extensions may be sutured to the sclera.

15. The method of claim 1, wherein the shaped conical inlay is of a suitable thickness to permit reshaping with a femtosecond laser after it has been attached to the human cornea so as to adjust the power of the corneal inlay.

16. The method of claim 1, wherein the step of placing the corneal tissue in a fixative solution for a selected time interval causes the corneal inlay to be able to be stored for a period of up to two years.
